(19) United States
(12) Patent Application Publication
(10) Pub. No.: US 2006/0257983 A1
(43) Pub. Date: Nov. 16, 2006

(54) Metabolically engineered micro-organisms having reduced production of undesired metabolic products

(75) Inventors: Christoffer Bro, Gentofte (DK); Brigitte Regenberg, Kobenhavn (DK); Jens Nielsen, Charlottenlund (DK)

Correspondence Address: BROWDY AND NEIMARK, P.L.L.C.;624 NINTH STREET, NW, SUITE 300, WASHINGTON, DC, 20001-5303, US

(73) Assignee: Fluxome Sciences A/S, Lyngby (DK)

(21) Appl. No.: 10/536,495

(22) PCT Filed: Nov. 25, 2003
(86) PCT No.: PCT/EP03/13231

§ 371 Date: May 10, 2006

(30) Foreign Application Priority Data
Nov. 25, 2002 (GB) 0227435.5

(51) Int. Cl. C12P 7/40; C12P 7/06; C12N 15/74; C12N 1/18

(52) U.S. Cl. 435/136; 435/161; 435/254.2; 435/483

(57) ABSTRACT

A metabolically engineered micro-organism has an operative first metabolic pathway in which a first metabolite is transformed into a second metabolite in a reaction in which NAD is a cofactor for a first enzyme, suitably a phosphorylating dehydrogenase, said reaction step producing NADH. Said second metabolite is transformed into at least one further metabolite in a reaction catalysed by a second enzyme, suitably a kinase. The organism has an operative second metabolic pathway characterised by an enzyme activity in excess of a native level in respect of a third enzyme, suitably a non-phosphorylating dehydrogenase, e.g. GAPN, catalysing a non-reversible reaction in which NADP is a cofactor and NADPH is a product. Said first metabolite is transformed into a said further metabolite without the involvement of said second enzyme.

In the metabolic production of low-value added products like ethanol, lactic acid, citric acid, amino acids and many antibiotics, the yield of product on the substrate, i.e. the amount of product formed per unit substrate consumed (often given as kg product formed per kg substrate consumed), and the productivity,. i.e. the amount of product formed per unit reaction volume and per unit time (often given as kg product formed per m³ reaction volume per hour) are the most important design variables to optimise. In order to obtain a high yield and productivity it is necessary to direct the carbon fluxes from the substrate towards the metabolite of interest at a high rate and at the same time minimize the formation of all possible by-products. This often requires engineering of the central carbon metabolism, which is difficult due to the tight regulation in this part of the cellular metabolism (Nielsen, 2001).

Many fermentation processes have been optimised such that the desired product is predominantly formed. However, due to the complexity of cellular metabolism it is inevitable that by-products are formed. This may be undesirable for at least three reasons:
The by-product(s) may be toxic to humans or animals.
The by-product(s) may cause problems in the subsequent separation process or represent an environmental burden
Formation of the by-product(s) results in a loss of carbon, and the overall yield of product on the raw material is therefore below the theoretical maximum.

The first reason is clearly problematic if humans or animals may be exposed to the product, either directly or indirectly. Typically, one chooses a cellular system that does not produce toxins when products are made for human consumption, e.g. fungal cells that do not produce aflatoxins are used for production of food grade enzymes, and E. coli strains that do not produce endotoxins are used for production of pharmaceuticals. The second reason may especially be problematic if the by-product has properties very similar to those of the desired product, since separation of the active product then requires very efficient separation principles, which are often costly. In some cases the by-product may also cause other types of problems, e.g. inactivation of the desired product. Problems with loss of carbon in the by-product is mainly a problem in connection with the production of low-value added products like ethanol, citric acid, amino acids and many antibiotics.

By way of example, a major problem in connection with ethanol production by anaerobic fermentation of S. cerevisiae is a substantial formation of glycerol as a by-product. Under anaerobic growth conditions cytosolic NADH formed from biomass formation can only be reconverted to NAD⁺ via glycerol formation (van Dijken and Scheffers, 1986). There are two genes, GPD1 and GPD2, encoding glycerol-3-phosphate dehydrogenase that regenerates NAD⁺ from NADH while converting dihydroxyacetone-phosphate to glycerol-3-phosphate. Disruption of GPD2 results in some reduction of glycerol formation, but the specific growth rate is also significantly reduced (Valadi et al., 1998; Nissen et al., 2000a). A double gpd1gpd2 deletion mutant strain is not able to grow at anaerobic conditions, and introduction of a new pathway to regenerate NAD⁺ was therefore attempted by expressing a bacterial transhydrogenase (catalysing: NADH+NADP⁺<=>NAD⁺+NADPH) of Azotobacter vinelandii in a double gpd1gpd2 deletion mutant strain (Nissen et al., 2000a). Expression of the transhydrogenase could, however, not restore growth under anaerobic conditions. Enhanced expression of a transhydrogenase is disclosed also in EP-A-0733712.

WO99/46363 report on the expression of a phosphorylating dehydrogenase resulting in a net transhydrogenase activity in living cells with the aim to improve product formation. Thus, through over-expression of GDH2, encoding a phosphorylating NADH-dependent glutamate dehydrogenase (EC 1.2.1.12), they attempted to increase the ethanol formation from xylose and glucose. Another attempt using the same concept was to express a fungal NADPH dependent glyceraldehyde dehydrogenase as described by Verho et al. (2002). However, during growth on xylose the recombinant cells also produced substantially more xylitol.

In another approach to improve ethanol production and decrease glycerol formation Nissen et al. (2000b) engineered the ammonia assimilation in S. cerevisiae. Through disruption of GDH1, encoding NADPH-dependent glutamate dehydrogenase, and over-expression of GDH2 the production of NADH in association with biomass synthesis was reduced significantly resulting in a more than 40% reduction of the glycerol yield (Nissen et al., 2000b). Furthermore, through over-expression of the GS-GOGAT pathway for ammonia assimilation, which is also NADH-dependent in S. cerevisiae, both a reduction in the glycerol yield and an increase in the ethanol yield was obtained (Nissen et al., 2000b). This increase in ethanol yield is due to the additional consumption of ATP in the GS-GOGAT pathway. This example illustrates that redirection of the fluxes through the central carbon metabolism can be obtained through engineering other parts of the metabolism, particularly through modulation of the redox metabolism, and this approach is likely to function also for improving the production of other products as the supply of NADPH for biosynthesis is often limiting the capacity for production. While NADPH is almost exclusively used as electron donor in biosynthesis in the cell, NADH is primarily used for generation of free energy (often in the form of high-energy phosphate-bonds in ATP). Under some conditions, such as anaerobic growth of S. cerevisiae on a fermentable sugar, surplus amounts of NADH are formed which cannot by used in generation of ATP, and this results in the formation of by-products, primarily glycerol.

Valverde et al (1999)discloses an E. coli strain engineered to express cDNA containing the Pisum sativum GapN gene which encodes the non-phosphorylating glyceraldehyde-3-phosphate dehydrogenase GAPN or GAPDHN (EC 1.2.1.9). The strain has its native Gap-2 gene encoding its NAD-dependent phosphorylating glyceraldehyde-3-phosphate dehydrogenase GAPDH disabled by an insertion. It is found that the expression of GAPN re-establishes the ability of the strain to grow aerobically on sugars, but the strain is still unable to perform anaerobic fermentation.

The present invention now provides a metabolically engineered micro-organism having an operative first metabolic pathway in which a first metabolite is transformed into a second metabolite in a reaction in which NAD is a cofactor for a first enzyme, said reaction step producing NADH, and in which said second metabolite is transformed into at least one further metabolite in a reaction catalysed by a second enzyme, and having an operative second metabolic pathway characterised by an enzyme activity in excess of a native level in respect of a third enzyme catalysing a non-reversible reaction in which NADP is a cofactor and NADPH is a product and in which said first metabolite is transformed into a said further metabolite without the involvement of said second enzyme.

In a micro-organism of the invention as described above, said first metabolic pathway is preferably a native pathway.

In certain preferred embodiments, said first enzyme is a phosphorylating dehydrogenase.

In certain preferred embodiments, including those referred to immediately above, said second enzyme is a kinase.

In certain preferred embodiments, including those referred to in the two paragraphs immediately above, said third enzyme is a non-phosphorylating dehydrogenase, for instance said third enzyme is GAPN (EC 1.2.1.9).

An example of a phosphorylating dehydrogenase first enzyme is GAPDH (EC 1.2.1.12).

In certain preferred embodiments, including all those referred to above, at least one copy of a genetic sequence encoding said third enzyme has been recombinantly introduced into said organism.

Preferably, a genetic sequence encoding said third enzyme is operatively linked to an expression signal not natively associated with said genetic sequence in said organism.

The micro-organism of the invention may preferably be a yeast. This may be an ethanol producing fermenting yeast. It may be a strain of Saccharomyces cerevisiae.

More generally, the micro-organism may be a species belonging to the genus Saccharomyces, e.g. S. cerevisiae, S. kluyveri, S. bayanus, S. exiguus, S. sevazzi, S. uvarum , a species belonging to the genus Kiyuveromyces, e.g. K. lactis K. marxianus var. marxianus, K. thermotolerans, a species belonging to the genus Candida, e.g. C. utilis C. tropicalis, a species belonging to the genus Pichia, e.g. P. stipidis, P. pastoris, P. sorbitophila, or other yeast species, e.g. Debaromyces hansenii, Hansenula polymorpha, Yarrowia lipolytica, Zygosaccharomyces rouxii or Schizosaccharomyces pombe.

Concerning other micro-organisms (non-yeast), a non-exhaustive list of suitable micro-organisms will include the following:
Escherichia coli, Corynebacterium glutamicum, Aspergillus niger, Aspergillus awamori, Aspergillus oryzae, Aspergillus nidulans, Penicillium chrysogenum, Rhizopus oryzae.

The invention includes a genetically transformed micro-organism containing one or more copies of an heterologous DNA sequence encoding GAPN operatively associated with an expression signal and having a functional native or heterologous expression capability for GAPDH (EC 1.2.12).

In a further aspect, the invention includes a method of producing a desired metabolic product with decreased production of an undesired metabolic product, comprising culturing a micro-organism of the invention as described above. The undesired metabolic product may be glycerol, acetate or an amino acid, but it may also be any other metabolite secreted by micro-organisms.

The desired product may be ethanol, lactic acid, citric acid, an amino acid or an antibiotic.

The invention will therefore be useful for the improvement of production of ethanol but also of metabolites other than ethanol in micro-organisms. Thus, Porro et al. (1999) describe production of lactic acid in yeast through deletion of pyruvate decarboxylase activity and expression of a heterologous activity of lactate dehydrogenase. In the conversion of pyruvate to lactate there is regeneration of NAD⁺ as is the case in the overall conversion of pyruvate to ethanol. The overall conversion of a sugar to lactic acid therefore has high similarity with the conversion of a sugar to ethanol, and the invention will consequently have a positive effect on lactic acid production.

In the production of citric acid by the filamentous fungus Aspergillus niger there is a net formation of NADH in the conversion of sugar to citric acid, i.e. NADH is generated at the location of glyceraldehydes-3-P dehydrogenase and pyruvate dehydrogenase. Through expression of GAPN it will be possible to replace part of the net formation of NADH with a net formation of NADPH, which is needed for protein synthesis. Hence, sugar that may otherwise be used to generate redox power in the form of NADPH, e.g. through the pentose phosphate pathway where sugar is converted to carbon dioxide with parallel formation of NADPH, may be redirected towards formation of citric acid resulting in a higher yield of the product on the sugar. Similar reasoning will hold for the production of other metabolites like succinic acid and malic acid.

In the production of many amino acids, e.g. lysine by Corynebacterium glutamicum, the amino acid is derived from precursor metabolites of the central carbon metabolism. Thus, in bacteria lysine is derived from oxaloacetate, which again is derived from pyruvate or phosphoenolpyruvate. In the conversion of sugar to the precursor metabolite there is a net production of NADH and the overall conversion of sugar to lysine therefore involves a net production of NADH. In the conversion of oxaloacetate to lysine there is a net consumption of NADPH (in some cases indirectly through the use of glutamate which needs to be regenerated from 2-oxoglutarate with expenditure of NADP). Expression of GAPN may therefore lead to a reduced net formation of NADH and a reduced net consumption of NADPH in the overall conversion of sugar to lysine. Similar reasoning will hold for the synthesis of other amino acids, e.g. isoleucine, threonine and phenylalanine.

In the production of many antibiotics, e.g. penicillin by Penicillium chrysogenum, there is also a net production of NADH and a net consumption of NADPH in the overall conversion of sugar to antibiotics. For these processes expression of GAPN will therefore also be beneficial.

The enhanced expression of GAPN may be beneficial in combination with modulated (enhanced or suppressed) expression or activity of one or more other enzymes. Mention has been made above of interaction between GAPN expression and expression of lactate dehydrogenase in yeast. Generally, the invention may be used to improve the metabolism of pentose sugars. The invention may be used to improve xylose uptake and to reduce xylitol secretion. In many microorganisms xylose metabolism involves xylose reductase (XR), which converts xylose to xylitol, xylitol dehydrogenase (XDH), which converts xylitol to xylulose, and finally xylulose kinase that phosphorylates xylulose to xylulose-5-phosphate which enters the pentose phosphate pathway. XR involves formation of NADPH (the enzyme may use both NAD⁺ and NADP⁺ as co-factor, but it has preference for NADP⁺) whereas XDH involves consumption of NADH. There is therefore a net consumption of NADPH and a net formation of NADH upon xylose uptake. Thus, increased GAPN expression may, if it is linked with expression of xylose reductase, xylose dehydrogenase, and xylitol kinase, result in an increased xylose uptake.

Expression of GAPN or another said third enzyme may be provided for by the introduction into a micro-organism of one or more copies of a DNA coding sequence for the enzyme either with an heterologous promoter or placed under the control of an native promoter sequence. Suitably, the coding sequence and an effective expression signal therefore is introduced in a multi-copy plasmid.

In preferred embodiments, this invention specifically targets the problem of production of surplus of NADH in functioning cells and the problem with a limited supply of NADPH. The cells are enabled to increase the formation of NADPH at the cost of NADH formation. This is done for example, by expression of a non-phosphorylating, NADP⁺-dependent glyceraldehyde-3-phosphate dehydrogenase (GAPN) (EC 1.2.1.9) in a cell, as a means to alter the redox metabolism in the cell.

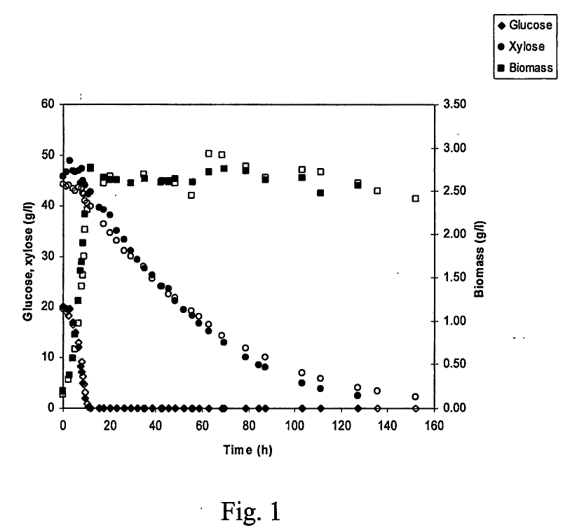

GAPN catalyses the irreversible oxidation of glyceraldehyde-3-phosphate and NADP⁺into 3-phosphoglycerate and NADPH. In most cells the conversion of glyceraldehyde-3-phosphate into 3-phosphoglycerate is only catalysed by the sequential action of two enzymes, i.e. NAD⁺-dependent glyceraldehyde-3-phosphate dehydrogenase (GAPDH) (EC 1.2.1.12) and phosphoglycerate kinase (PGK) (EC 2.7.2.3) by conversion of NAD⁺ and ADP into NADH and ATP (see FIG. 1). The net stoichiometry of the routes converting glyceralde-3-P into 3-P-glycerate are:
GAPN: glyceraldehyde-3-phosphate+NADP⁺→3-phosphoglycerate+NADPH
GAPDH+PGK: glyceraldehyde-3-phosphate+NAD⁺+ADP+P=3-phosphoglycerate+NADH+ATP

The reaction catalysed by GAPN thereby yields one NADPH instead of one NADH and one ATP when comparing with the total reaction catalysed by GAPDH and PGK.

The conversion of glyceraldehyde-3-phosphate into 3-phosphoglycerate is part of the glycolysis, which is the main energy-yielding pathway, and the reaction is therefore always active when a cell is growing on hexose or pentose containing substrates.

By controlling the amount of gapN expressed in the microbial cell control can be exercised over how large a part of the conversion of glyceraldehyde-3-phosphate into 3-phosphoglycerate should go through GAPN. Thereby the redox metabolism can be manipulated by controlling the amount of one NAPDH formed at the expense of one NADH and ATP.

The Examples below make reference to the accompanying drawings in which:

FIG. 1 shows the results obtained in Example 8 in respect of the profiles of biomass, glucose and xylose concentrations (The symbols for xylose are filled circles and hollow diamonds and the two curves lie close overlapping one another); and

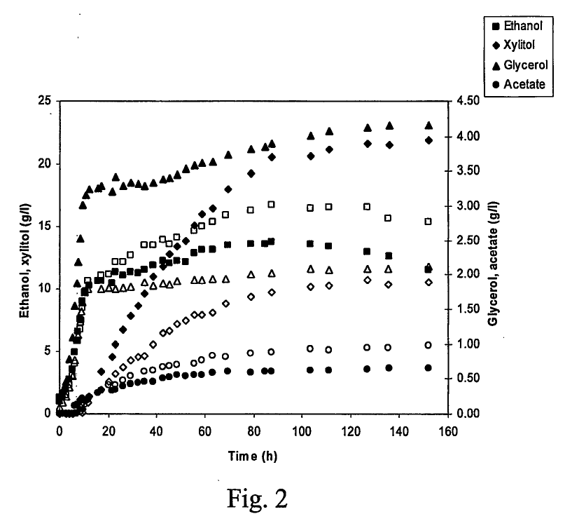

FIG. 2 shows the results obtained in Example 8 in respect of the profiles of ethanol, xylitol, glycerol and acetate concentrations. (Closed and open square symbols are used for ethanol and for acetate. The lower closed and open square defined curves are acetate. The upper closed and open square defined curves are ethanol.)

To illustrate this in the Examples below, a nucleotide sequence containing the non-phosphorylating, NADP⁺-dependent glyceraldehyde-3-phosphate dehydrogenase gene (gapN) from Streptococcus mutans was expressed in S. cerevisiae on a multicopy plasmid. The resulting strain was characterised in anaerobic batch cultivations on the hexose glucose—a typical method for ethanol production (Example 3) and on xylose (Example 8). NADP⁺-dependent glyceraldehyde-3-phosphate dehydrogenase activity was determined in Example 5 in both the gapN strain of Example 1 and in a reference strain carrying the empty plasmid. Activity of GAPN could only be measured in the gapN strain and the activity was approximately 10% of that of the NAD⁺-dependent glyceraldehyde-3-phosphate dehydrogenase activity.

The growth rate of the gapN strain of Example 1 was not affected by the expression of GAPN activity when comparing to the strain containing the empty plasmid. The gapN strain produced 43% less glycerol and 3% more ethanol. A much greater increase in ethanol production is achieved in Example 8.

Glycerol is formed by S. cerevisiae during anaerobic growth to maintain the cytosolic redox balance. Under anaerobic conditions NADH, produced as a result of production of biomass and organic acids, can only be oxidised to NAD⁺ by formation of glycerol, since respiration is not possible and the formation of ethanol is a redox-neutral process. The formation of glycerol is therefore a redox problem, so by introducing gapN into S. cerevisiae the production of glycerol will be reduced by one molecule for each molecule of glyceraldehyde-3-phosphate that is converted via GAPN. By having a flux through GAPN that is high enough the production of glycerol can be completely eliminated and the flux redirected to ethanol and/or biomass thereby increasing the ethanol yield.

By increasing the amount of NADPH for biosynthesis by expressing gapN in a cell it can potentially be possible to increase the production of a product such as protein where the supply of NADPH might be limiting.

Advantages of the exemplified approach include:
Larger changes in yields can be achieved.
Only one genetic change is required.
The activity of the enzyme only affects the specific reaction it catalysed and the redox metabolism—there are no effects on other parts of metabolism and therefore the growth rate is not affected.
By-production of glycerol in production of ethanol with yeast can be eliminated.

### EXAMPLE 1

Expression of GAPN in S. cerevisiae Strains: S. cerevisiae (M4054, S288C MATa ura3 gap1) was used for construction of a reference strain and GAPN strain. For long-term maintenance plasmid baring strains were grown to stationary phase in shake flask cultures on minimal media (see below). After addition of sterile glycerol to a concentration of 20% (vol/vol), aliquots were stored at −80° C. These frozen stocks were used for obtaining single colonies on plates with a minimal medium (Verduyn et al., 1990), which were stored at 4° C., and used within 2 weeks for inoculation of precultures.

Construction of reference strain: The empty pYX212 2μ high-copy vector containing the URA3 gene and the TPI1 promoter was transformed into S. cerevisiae (M4054) by electroporation.

Construction of GAPN strain: gapN was expressed on a pYX212 2μ high-copy vector containing the URA3 gene and the TPI1 promoter. The plasmid was constructed directly in S. cerevisiae (M4054) by cotransformation and homologous recombination between EcoRI digested pYX212 and PCR-amplified gapN from Streptococcus mutans. PCR was performed on genomic DNA from Streptococcus mutans using Expand High Fidelity (Roche) and one primer identical to the TPI1 promoter in pYX212 plus the first part of gapN (gapN-START-EcoRI-TPI promoter 5′-CTA CAA AAA ACA CAT ACA GGA ATT CAT GAC AAA ACA ATA TAA AAA TTA TG) and a second primer (gapN-STOP-NcoI-BamHI-AvrII-ApaI 5′-GGG CCC TAG GAT CCA TGG TGA ATT TTA TTA TTT GAT ATC AAA TAC GAC GG) identical to the MCS of pYX212 and the last part of gapN including the stop codon. Hence the ORF of gapN has been cloned between EcoRI and NcoI side in pYX212, down stream the TPI1 promoter. The original start codon TTG was substituted with an ATG, to make translation in S. cerevisiae possible. Construction was verified by diagnostic PCR.

### EXAMPLE 2

Shake flask cultivations and precultures: Aerobic shake flask cultivations were performed in baffled, cotton-stopped, 500 ml Erlenmeyer flasks to screen transformants obtained. Precultures for anaerobic batch cultivations were grown I similar flasks but without baffles. These flasks contained each 100 ml of a defined mineral medium containing 7.5 g/L (NH₄)₂SO₄; 14 g/L KH₂PO₄; 0.5 g/L MgSO₄, 7H₂O; 50 μl/L antifoam (Sigma A-8436); 2% (w/vol) glucose; trace metals (15 mg/L EDTA; 4.5 mg/L ZnSO₄, 7H₂O; 0.84 mg/L MnCl₂, 2H₂O; 0.30 mg/L CoCl₂, 6H₂O; 0.30 mg/L CuSO₄, H₂O; 0.40 mg/L Na₂MoO₄, 2H₂O; 4.5 mg/L CaCl₂, 2H₂O; 3.0 mg/L FeSO₄, 7H₂O; 1.0 mg/L H₃BO₃; and 0.10 mg/L KI) and vitamins (0.05 mg/L D(−)biotin; 1.0 mg/L Ca D(+)panthotenate; 1.0 mg/L nicotinic acid; 25 mg/L myo-inositol; 1.0 mg/L thiamine chloride hydrochloride; 1.0 mg/L pyridoxol hydrochloride; and 0.20 mg/L p-aminobenzoic acid). The pH of the mineral medium was set to 6.5 with NaOH and autoclaved separately from the glucose solution. After autoclavation the vitamin solution was added to the flasks by sterile filtration. Shake flasks and precultures were inoculated with a single colony from plate cultures a grown at 30° C. and 150 rpm. Precultures were grown to exponential phase and used for inoculation of anaerobic batch cultivations to a start concentration of 1 mg CDW/L. Growth of both the reference and the GAPN-strain was observed.

### EXAMPLE 3

Anaerobic batch cultivations: Cultivations were carried out in well-controlled laboratory fermentors (B. Braun Biotech, Germany) with a working volume of 2 litres. A defined mineral medium (Verduyn et al., 1990) was used, which contained per litre: 40 g glucose; 5.0 g (NH₄)₂SO₄; 3.0 g KH₂PO₄; 0.5 g MgSO₄, 7H₂O; and trace metals and vitamins as described in shake flask cultivations and precultures. 300 μl/L antifoam (Sigma A-8436) was added to avoid foaming and the medium was supplemented with 420 mg/L Tween 80 and 10 mg/L ergosterol, which is necessary for anaerobic growth of S. cerevisiae. The glucose solution was autoclaved separately from the mineral medium and afterwards added to the fermentor together with a sterile filtrated solution containing the vitamins and together with the Tween 80 and ergosterol, which first were dissolved in boiling pure ethanol.

Cultivations were carried out at 30° C. with a stirrer speed of 600 rpm and were flushed with nitrogen gas at a flow rate of 400 ml per minute. To minimise the diffusion of O₂ into the cultures, the bioreactors were fitted with Norprene tubing. The concentration of dissolved oxygen was measured with Mettler Toledo polarographic electrode and remained below the detection limit. pH was kept at 5.0 by automatic addition of 4 M KOH. The bioreactors were fitted with cooled condensers, and the off-gas was led to a gas analyser (INNOVA, Denmark) to measure the content of CO₂. The biomass concentration during fermentation with the reference and GAPN strains are shown in FIG. 2. It is found that the maximum specific growth rate of the GAPN strain is identical with that of the reference strain.

### EXAMPLE 4

Analysis of extracellular metabolites: Culture samples for determination of glucose, ethanol, glycerol, acetate, pyruvate and succinate concentrations were filtered through a 0.45 μm cellulose acetate filter (Osmonics) immediately after sampling, and the filtrate was frozen at −20° C. until further analysis. The concentrations of the metabolites were determined by high-pressure liquid chromatography on an Aminex HPX-87Hm column (Bio-Rad) kept at 65° C. and eluted at 0.6 ml per minute with 5 mM H₂SO₄. Acetate and pyruvate were detected spectrophotometrically by a Waters 486 Turnable Absobance Detector at 210 nm. Glucose, ethanol, glycerol and succinate were detected refractometrically by a Waters 410 Differential Refractometer. Measurement of the metabolites during anaerobic fermentations is shown in FIG. 3. The final concentrations of the metabolites are listed below. It is seen that the GAPN strain produces more ethanol and less glycerol.

| | Ethanol | Glycerol | Pyruvate |
|---|---|---|---|
| | (g/L) | (g/L) | (g/L) |
| Reference strain | 14.90 | 2.81 | 0.061 |
| GAPN strain | 17.05 | 2.06 | 0.066 |

Based on the measurements of glucose the overall conversion yields to the different metabolites were calculated and they are given below.

| | Ethanol (g/g | Glycerol (g/g | Pyruvate (g/g |
|---|---|---|---|
| | glc) | glc) | glc) |
| Reference | 0.392 | 0.078 | 0.0013 |
| strain | | | |
| GAPN strain | 0.403 | 0.0463 | 0.0012 |

It is observed that the ethanol yield is improved 3% and the glycerol yield is reduced by 40%.

### EXAMPLE 5

Measurement of enzyme activities: Cell free extracts were produced with the help of a Fastprep FP120 instrument (Savant Instruments, New York) as described by Møller et al. (2002).

Enzyme activities were assayed at 30° C. by following the NADPH or NADH production at 340 nm using a spectrophotometer (HP 8353 UV-VIS system with Chemstation software from Hewlett Packard). Glyceraldehyde-3-phosphate dehydrogenase activity was determined as described by Crow and Wittenberger (1979) in a 1 ml reaction mixture activity. For determination of non-phosphorylating, NADP⁺-dependent glyceraldehyde-3-phosphate dehydrogenase activity the reaction mixture contained: 125 mM triethanolamine/HCl buffer (pH 8.3), 1 mM NADP⁺, 5 mM 2-mercaptoethanol, and cell free extract. NAD⁺-dependent glyceraldehyde-3-phosphate dehydrogenase activity was determined with a reaction mixture containing: 125 mM triethanolamine/HCl buffer (pH 8.3), 1 mM NAD⁺, 5 mM cystein/HCl, and cell free extract. The reactions were started by adding DL-glyceraldehyde-3-phosphate (prepared from DL-glyceraldehyde-3-phosphate diethyl acetal, Sigma G-5376) to a final concentration of 2 mM. Protein content in cell free extracts was determined by the Lowry method, using fatty-acid free BSA (Sigma A-6003) as standard. Results from analysis of the enzyme activity are given below. It is seen that in the reference strain there is no activity of a NADP-dependent glyceraldehydes dehydrogenase whereas in the GAPN strain there is some low activity (accounting for about 10% of the total glyceraldehydes dehydrogenase activity).

| | Reference | |
|---|---|---|
| | strain | GAPN strain |
| NAD-dependent | 11.2 ± 0.74 | 7.03 ± 0.71 |
| glyceraldehyde | | |
| dehydrogenase | | |
| NADP-dependent | 0 | 0.65 ± 0.03 |
| glyceraldehyde | | |
| dehydrogenase | | |

### EXAMPLE 6

### Sequencing of GapN

In order to verify that no mutations had occurred in the gapN encoding gene expressed in the 2μ high copy number plasmid, the part of the plasmid containing the gapN was sequenced. The sequence of the gapN as inserted into the plasmid and as found actually to be present therein was ‘atg aca aaa caa tat aaa aat tat gtc aat ggc gag tgg aag ctt tca gaa aat gaa att aaa atc tac gaa ccg gcc agt gga gct gaa ttg ggt tca gtt cca gca atg agt act gaa gaa gta gat tat gtt tat gct tca gcc aag aaa gct caa cca gct tgg cga tca ctt tca tac ata gaa cgt gct gcc tac ctt cac aag gta gca gat att ttg atg cgt gat aaa gaa aaa ata ggt gct gtt ctt tcc aaa gag gtt gct aaa ggt tat aaa tca gca gtc agc gaa gtt gtt cgt act gca gaa atc att aat tat gca gct gaa gaa ggt ctt cgt atg gaa ggt gaa gtc ctt gaa ggc ggc agt ttt gaa gca gcc agc aag aaa aaa att gcc gtt gtt cgt cgt gaa cca gta ggt ctt gta tta gct att tca cca ttt aac tac cct gtt aac ttg gca ggt tcg aaa att gca ccg gct ctt att gcg gga aat gtt att gct ttt aaa cca ccg acg caa gga tca atc tca ggg ctc tta ctt gct gaa gca ttt gct gaa gct gga ctt cct gca ggt gtc ttt aat acc att aca ggt cgt ggt tct gaa att gga gac tat att gta gaa cat caa gcc gtt aac ttt atc aat ttt act ggt tca aca gga att ggg gaa cgt att ggc aaa atg gct ggt atg cgt ccg att atg ctt gaa ctc ggt gga aaa gat tca gcc atc gtt ctt gaa gat gca gac ctt gaa ttg act gct aaa aat att att gca ggt gct ttt ggt tat tca ggt caa cgc tgt aca gca gtt aaa cgt gtt ctt gtg atg gaa agt gtt gct gat gaa ctg gtc gaa aaa atc cgt gaa aaa gtt ctt gca tta aca att ggt aat cca gaa gac gat gca gat att aca ccg ttg att gat aca aaa tca gct gat tat gta gaa ggt ctt att aat gat gcc aat gat aaa gga gcc act gcc ctt act gaa atc aaa cgt gaa ggt aat ctt atc tgt cca atc ctc ttt gat aag gta acg aca gat atg cgt ctt gct tgg gaa gaa cca ttt ggt cct gtt ctt ccg atc att cgt gtg aca tct gta gaa gaa gcc att gaa att tct aac aaa tcg gaa tat gga ctt cag gct tct atc ttt aca aat gat ttc cca cgc gct ttt ggt att gct gag cag ctt gaa gtt ggt aca gtt cat atc aat aat aag aca cag cgc ggc acg gac aac ttc cca ttc tta ggg gct aaa aaa tca ggt gca ggt att caa ggg gta aaa tat tct att gaa gct atg aca act gtt aaa tcc gtc gta ttt gat atc aaa', which is identical with the sequence of gapN from Streptococcus mutans.

### EXAMPLE 7

### Expression of GAPN in a Xylose Metabolising Strain of S. cerevisiae

Strains: S. cerevisiae (MATa SUC2 MAL2-8 pADH-XYL1 pPGK-XYL2 pPGK-XKS1 ura3⁻) was used for construction of a xylose metabolising strain of S. cerevisiae expressing GAPN. For long-term maintenance, plasmid bearing strains were grown to stationary phase in shake flask cultures on minimal media (see below). After addition of sterile glycerol to a concentration of 20% (vol/vol), aliquots were stored at −80° C. These frozen stocks were used for obtaining single colonies on plates with a minimal medium (Verduyn et al., 1990), which were stored at 4° C., and used within 2 weeks for inoculation of precultures.

Construction of reference strain: The empty pYX212 2μ high-copy vector containing the URA3 gene and the TPI1 promoter was transformed into S. cerevisiae (MATa SUC2 MAL2-8 pADH-XYL1 pPGK-XYL2 pPGK-XKS1 ura3⁻) by the LiAc method and positive clones, containing the pYX212 URA3 2μ plasmid, were selected on synthetic minimal ammonium medium with glucose. Construction of GAPN strain: The pYX212 2μ high-copy vector containing the URA3 gene and the TPI1 promoter in front of the gapN encoding gene was transformed into S. cerevisiae (MATa SUC2 MAL2-8 pADH-XYL1 pPGK-XYL2 pPGK-XKS1 ura3⁻) by the LiAc method, and positive clones, containing gapN down stream the TPI1 promoter in the URA3 2μ plasmid, were selected on synthetic minimal ammonium medium with glucose.

A number of transformants was tested in aerobic shake flasks on glucose for growth rate and GAPN activity. A transformant was chosen with unchanged growth rate and high GAPN activity and denoted Xyl GAPN strain—the reference strain was denoted Xyl reference strain. Both strains had similar xylose reductase and xylose dehydrogenase activities, which were within the expected range.

### EXAMPLE 8

Anaerobic batch cultivations of a xylose metabolising strain of Saccharomyces cerevisiae: Cultivations were carried out in well-controlled laboratory fermentors with a working volume of 4 litres. A defined mineral medium (Verduyn et al., 1990) was used, which contained per litre: 20 g glucose; 50 g xylose; 5.0 g (NH₄)₂SO₄; 3.0 g KH₂PO₄; 0.5 g MgSO₄, 7H₂O; and trace metals and vitamins as described in shake flask cultivations and precultures. 500 μl/L antifoam (Sigma A-8436) was added to avoid foaming and the medium was supplemented with 420 mg/L Tween 80 and 10 mg/L ergosterol, which is necessary for anaerobic growth of S. cerevisiae. The glucose solution was autoclaved separately from the mineral medium and afterwards added to the fermentor together with a sterile filtered solution containing the vitamins and together with the Tween 80 and ergosterol, which first were dissolved in boiling pure ethanol.

Cultivations were carried out at 30° C. with a stirrer speed of 500 rpm and were flushed with nitrogen gas at a flow rate of 800 ml per minute. To minimise the diffusion of O₂ into the cultures, the bioreactors were fitted with Norprene tubing. pH was kept at 5.0 by automatic addition of 2 M NaOH. The bioreactors were fitted with cooled condensers, and the off-gas was led to a gas analyser (INNOVA, Denmark) to measure the content of CO₂. The profiles of the concentrations of biomass, glucose and xylose for both the Xyl reference strain (closed symbols) and the Xyl GAPN strain (open symbols) are shown in FIG. 1. The profiles of the concentrations of ethanol, xylitol, glycerol and acetate for both the Xyl reference strain (closed symbols) and the Xyl GAPN strain (open symbols) are shown in FIG. 2. It is found that the glucose and xylose uptake is approximately the same in the two strains whereas the Xyl GAPN strain produces much more ethanol, much less xylitol and less glycerol compared with the Xyl reference strain. The Xyl GAPN strain produces slightly more acetate than the Xyl reference strain. After 50 hours of fermentation the following concentrations of the metabolic products were obtained:

| | Ethanol | Glycerol | Xylitol |
|---|---|---|---|
| | (g/L) | (g/L) | (g/L) |
| Xyl reference strain | 12.3 | 3.5 | 13.4 |
| Xyl GAPN strain | 14.1 | 1.9 | 7.2 |

This corresponds to a 15% increase in the ethanol concentration, a 45% decrease in glycerol concentration and a 45% decrease in xylitol concentration.

During growth on glucose there is a slightly higher production of ethanol and a smaller production of glycerol by the Xyl GAPN strain (in analogy with results of Example 4). During growth on xylose overall the yield coefficients were estimated and are given below.

| | Ethanol | Glycerol | Xylitol |
|---|---|---|---|
| | (g/g sugar) | (g/g sugar) | (g/g sugar) |
| Xyl reference strain | 0.22 | 0.065 | 0.33 |
| Xyl GAPN strain | 0.29 | 0.037 | 0.17 |

These yield coefficients corresponds approximately with a 30%. increase in the ethanol yield, a 40% decrease in glycerol yield and an almost 50% decrease in xylitol yield.

### EXAMPLE 9

### Anaerobic Chemostat Cultivations of a Xylose Metabolising Strain of Saccharomyces cerevisiae

Anaerobic steady-state chemostat cultures were obtained in well-controlled 2-litre jacketed bioreactors (B. Braun Biotech, Melsungen, Germany) with a constant working volume of 1.0 litre and a dilution rate of 0.05 h⁻¹ (which equals the specific growth rate). Cultivations were carried out at 30° C. with a stirrer speed of 500 rpm and were flushed with pure nitrogen gas (0.2 liter·min⁻¹) . pH was kept at 5.0 by automatic addition of 2 M NaOH. To minimise the diffusion of O₂ into the cultures, the bioreactors were fitted with Norprene tubing. The concentration of dissolved oxygen was measured with Mettler Toledo polarographic electrode and remained below the detection limit. The bioreactors were fitted with cooled condensers (5° C.), and the off-gas was led to a gas analyser (INNOVA, Denmark) to measure the content of CO₂. Steady state was assumed when at least five residence times had passed since starting the continuous cultivation and was verified by constant (±2%) biomass concentration, metabolite concentrations, and carbon dioxide evolution rate at two measurements taken over 2 residence times. The cultures were fed with a defined medium similar to that described in Example 8.

Precultures for chemostat cultivations were grown at 30° C. and 150 rpm for 16-24 hours in cotton-stopped, 500 ml Erlenmeyer flasks with baffles containing each 100 ml of a media of pH 6.5 similar to that in the fermenters, except from different concentrations of glucose (20 g·l⁻¹), (NH₄)₂SO₄ (7.5 g·l⁻¹), KH₂PO₄ (14 g·l⁻¹), and antifoam (50 μl·l⁻¹), and furthermore, no Tween or ergosterol were added. Precultures were inoculated from plate cultures grown at 30° C. Continuous cultures were inoculated with 20 ml exponential growing preculture.

In the chemostat culture at steady state the concentrations of ethanol, glycerol and xylitol were measured, and the results are given below.

| | Ethanol | Glycerol | Xylitol |
|---|---|---|---|
| | (g/L) | (g/L) | (g/L) |
| Xyl reference strain | 10.84 | 1.49 | 7.41 |
| Xyl GAPN strain | 12.05 | 0.55 | 4.53 |

Based on the measurements of sugars the overall conversion yields to the different metabolites were calculated and they are given below.

| | Ethanol | Glycerol | Xylitol |
|---|---|---|---|
| | (g/g sugar) | (g/g sugar) | (g/g sugar) |
| Xyl reference | 0.29 | 0.044 | 0.21 |
| strain | | | |
| Xyl GAPN strain | 0.36 | 0.018 | 0.14 |

It is observed that the ethanol yield is improved by 24% and the glycerol yield is reduced by almost 60% and that the xylitol yield is reduced by 33%.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’.

### REFERENCES

Nielsen, J. (2001). Metabolic engineering. Appl. Microbiol. Biotechnol. 55, 263-283.
Nissen, T. L., Hamann, C. W., Kielland-Brandt, M. C., Nielsen, J., and Villadsen, J. (2000a). Anaerobic and aerobic batch cultivations of Saccharomyces cerevisiae mutants impaired in glycerol synthesis. Yeast 16, 463-474.
Nissen, T. L., Kielland-Brandt, M. C., Nielsen, J., and Villadsen, J. (2000b). Optimization of ethanol production in Saccharomyces cerevisiae by metabolic engineering of the ammonium assimilation. Metabol. Eng. 2, 69-77.
Porro, D., Bianchi, M. M., Brambilla, L., Menghini, R., Bolzani, D., Carrera; V., Lievenze, J., Liu, C. L., Ranzi, B. M., Frontali, L., and Alberghina, L. (1999). Replacement of a metabolic pathway for large-scale production of lactic acid from engineered yeasts. Appl. Environ. Biotechnol. 65, 4211-4215.
Valadi, H., Larsson, C., and Gustafsson, L. (1998). Improved ethanol production by glycerol-3-phosphate dehydrogenase mutants of Saccharomyces cerevisiae. Appl. Microbiol. Biotechnol. 50, 434-439.
Valverde, F., Losada, M., and Serrano, A. (1999). Engineering a central metabolic pathway: glycolysis with no net phosphorylation in an Escherichia coli gap mutant complemented with a plant GapN gene. FEBS Lett. 449, 153-158.
Van Dijken, J. P. and Scheffers, W. A. (1986). Redox balances in the metabolism of sugars by yeasts. FEMS Microbiol. Rev. 32, 199-224.
Verduyn, C., Postma, E., Scheffers, W. A., and van Dijken, J. P. (1990). Physiology of Saccharomyces cerevisiae in anaerobic glucose-limited chemostat cultures. J. Gen. Microbiol. 136, 395-403.
Verho, R., Richard, P., Jonson, P. H., Sundqvist, L., Londesborough, J., and Penttilä, M. (2002) Identification of the first fungal NADP-GAPDH from Kluyveromyces lactis. Biochem. 41, 13833-13838

1. A metabolically engineered micro-organism which is yeast having an operative first metabolic pathway in which a first metabolite is transformed into a second metabolite in a reaction in which NAD is a cofactor for a first enzyme, said reaction step producing NADH, and in which said second metabolite is transformed into at least one further metabolite in a reaction catalysed by a second enzyme, and having an operative second metabolic pathway characterized by an enzyme activity in excess of a native level in respect of a third enzyme catalyzing a non-reversible reaction in which NADP is a cofactor and NADPH is a product and in which said first metabolite is transformed into a said further metabolite without the involvement of said second enzyme.

2. A micro-organism as claimed in claim 1, wherein said first metabolic pathway is a native pathway.

3. A micro-organism as claimed claim 1, wherein said first enzyme is a phosphorylating dehydrogenase.

4. A micro-organism as claimed in claim 1, wherein said second enzyme is a kinase.

5. A micro-organism as claimed in claim 3, wherein said third enzyme is a non-phosphorylating dehydrogenase.

6. A micro-organism as claimed in claim 5, wherein said third enzyme is GAPN (EC 1.2.1.9).

7. A micro-organism as claimed in claim 6, wherein said first enzyme is GAPDH (EC 1.2.1.12).

8. A micro-organism as claimed in claim 1, wherein at least one copy of a genetic sequence encoding said third enzyme has been recombinantly introduced into said organism.

9. A micro-organism as claimed in claim 1, wherein a genetic sequence encoding said third enzyme is operatively linked to an expression signal not natively associated with said genetic sequence in said organism.

10. (canceled)

11. A micro-organism as claimed in claim 1, which is a yeast belonging to the genus Saccharomyces, Klyuveromyces, Candida, Pichia, Debaromyces, Hansenula, Yarrowia, Zygosaccharomyces or Schizosaccharomyces.

12. A micro-organism as claimed in claim 11, which is a strain of Saccharomyces cerevisiae, S. kluyveri, S. bayanus, S. exiguus, S. sevazzi, S. uvarum, Klyuveromyces lactis K. marxianus var. marxianus, K. thermotolerans, Candida utilis C. tropicalis, Pichia stipidis, P. pastoris, P. sorbitophila, Debaromyces hansenii, Hansenula polymorpha, Yarrowia lipolytica, Zygosaccharomyces rouxii or Schizosaccharomyces pombe.

13. A genetically transformed micro-organism which is a yeast containing one or more copies of a heterologous DNA sequence encoding GAPN (EC 1.2.1.9) operatively associated with an expression signal and having a functional native or heterologous expression capability for GAPDH (EC 1.2.12).

14. A method of producing a desired metabolic product with decreased production of an undesired metabolic product, comprising culturing a micro-organism as claimed in claim 1 wherein the desired metabolic product is the product of the reaction catalyzed by said third enzyme, and the undesired metabolic product is the product of the reaction catalyzed by the second enzyme.

15. A method as claimed in claim 14, wherein the desired product is ethanol, lactic acid, citric acid, an amino acid or an antibiotic.

16. A method as claimed in claim 14, wherein said undesired metabolic product is glycerol, acetate or an amino acid.
